(19) United States
(12) Patent Application Publication
(10) Pub. No.: US 2025/0000579 A1
(43) Pub. Date: Jan. 2, 2025

(54) PHOTIMECHANICAL VASCULAR THERAPY

(71) Applicant: KONINKLIJKE PHILIPS N.V., EINDHOVEN (NL)

(72) Inventors: Cedar Decker, ALBERTVILLE, MN (US); Sooneon Bae, MAPLE GROVE, MN (US); Andy Scherer, MAPLE GROVE, MN (US)

(21) Appl. No.: 18/710,648

(22) PCT Filed: Dec. 5, 2022
(86) PCT No.: PCT/EP2022/084328

§ 371 Date: May 16, 2024

Related U.S. Application Data
(60) Provisional application No. 63/288,726, filed on Dec. 13, 2021.

(51) Int. Cl. A61B 18/24; A61B 17/00; A61B 17/221; A61B 18/00; A61B 18/22

(52) U.S. Cl. CPC A61B 18/245; A61B 2017/00867; A61B 2017/2215; A61B 2018/0041; A61B 2018/00577; A61B 2018/2211

(57) ABSTRACT

A thrombectomy or atherectomy device (10) includes a catheter (12); a capture device (22, 22₁) disposed at a distal end of the catheter; and a laser aperture (24, 24₁) disposed proximate to the capture device and configured to ablate material of a clot (C) in a blood vessel (V) of a patient that is captured by the capture device.

### FIELD

The following relates generally to the intravascular therapy instrument arts, thrombectomy arts, atherectomy arts, intravascular laser ablation arts, and related arts.

### BACKGROUND

Venous thromboembolism, which includes deep venous thrombosis (DVT), is a major contributor disease, and is the third most common cardiovascular pathology after coronary artery disease and stroke. Lower extremity DVT (LEDVT) can block the venous lumen and leads to venous congestion, swelling, and lower extremity venous valve function damage, resulting in post-thrombotic syndrome (PTS).

Standard treatment of venous obstructions include the use of balloons, stents, lytics, and mechanical aspiration. Balloons and stents are inexpensive and time efficient treatment options but do not remove the obstruction from the vessel, which can lead to reoccurrence of the disease. Lytics usually require 24 hours of administration and often require an adjunctive procedure to fully relieve patient symptoms. Additionally, the effectiveness of lytics drops off significantly with the age of the clot. Mechanical thrombectomy treatments involve collection and extraction of the clot, either by mechanical entanglement and transport, or through use of vacuum aspiration. Typical complications associated with mechanical thrombectomy include emboli generation, blood loss, limited effectiveness in organized (i.e., old) clot, and poor interaction with previously placed stents.

Other types of intravascular therapy devices utilize a mechanical cutter or laser ablation to cut away clot material. These devices have a short working range: a mechanical cutter must physically contact the clot to remove material, while a typical laser ablation aperture has a working distance for ablating tissue of at most a few tens of microns. Hence, these devices typically have complex catheter tip rotation and bending mechanisms to steer the mechanical cutter or laser aperture into close proximity or direct contact with clot material. Cutting or ablating approaches are especially difficult in the venous system due to the typically larger diameter of veins compared with arteries and the typically less organized nature of venous clots.

The following discloses certain improvements to overcome these problems and others.

### SUMMARY

In some embodiments disclosed herein, a thrombectomy or atherectomy device includes a catheter; a capture device disposed at a distal end of the catheter; and a laser aperture disposed proximate to the capture device and configured to ablate material of a clot in a blood vessel of a patient that is captured by the capture device.

In some embodiments disclosed herein, a thrombectomy or atherectomy method includes inserting a catheter carrying an expandable capture device into a blood vessel to deliver the capture device to a clot disposed on an inner wall of the blood vessel; deploying the expandable capture device from a lumen of the catheter; retrieving the deployed expandable capture device back into the lumen of the catheter; and during the deploying and/or during the retrieving, ablating material of the clot that is captured by the capture device with laser light emitted by a laser aperture disposed at a distal end of the catheter or on the capture device.

In some embodiments disclosed herein, a thrombectomy or atherectomy device includes an intravascular clot retrieval device configured to mechanically transport clot material to a lumen of the intravascular clot retrieval device; and a laser aperture arranged to ablate the clot material as it is mechanically transported to the lumen of the intravascular clot retrieval device.

One advantage resides in reducing the risk of emboli generation and blood loss associated with existing mechanical thrombectomy treatments/devices and improve effectiveness in older, more organized, collagenous clot.

Another advantage resides in using laser energy with a clot collection mechanism to fully ablate the clot, rather than extract it from the patient.

Another advantage resides in reducing a risk of emboli generation during a capturing and extracting process of a clot in a blood vessel.

Another advantage resides in reducing blood loss during a vascular therapy procedure.

Another advantage resides in increasing an effectiveness of laser energy during a vascular therapy procedure by bringing a clot material in proximity to a laser source.

Another advantage resides in drawing clot material to a laser aperture for ablation, rather than steering the laser aperture to the clot disposed on the blood vessel wall.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a thrombectomy or atherectomy device in accordance with the present disclosure.

FIGS. 2A, 2B, 2C, and 2D diagrammatically illustrate the device of FIG. 1 in use.

FIG. 3 diagrammatically illustrates another embodiments of the device of FIG. 1.

FIG. 4 diagrammatically illustrates a method of performing a thrombectomy and atherectomy using one of the devices of FIG. 1 or 2.

### DETAILED DESCRIPTION

Presently, thrombectomies are typically done using cutting/aspiration or using an extraction device, while atherectomies are typically done using laser atherectomy. This is in part because the arteries tend to be smaller and arterial blockages more organized, thus making laser atherectomy more feasible than using a laser in a thrombectomy. However, thrombectomies are vulnerable to clot fragments being released during the capture process. Such clot fragments can embolize and create serious health risk.

The following discloses a combination of a capture device with a laser for ablating the clot as it is drawn into the catheter. To this end, the capture device is an expandable structure of Nitinol or the like, and a laser ring is disposed on the edge of the sheath. Advantageously, as the capture device with the attached clot material is withdrawn back into the sheath the attached clot material is drawn into the working distance of the lasers (on the order of 50 microns in some embodiments) and is ablated.

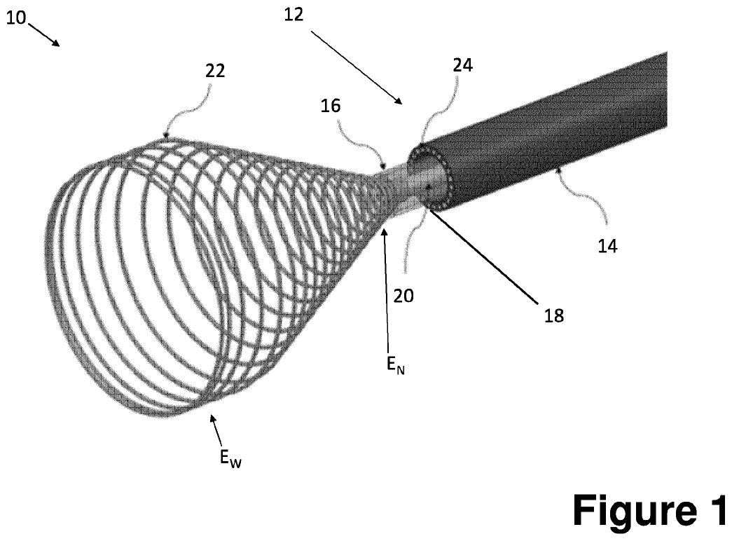

With reference to FIG. 1, a distal end of an illustrative intravascular therapy (e.g. thrombectomy or atherectomy) device 10 is shown. As shown in FIG. 1, the device 10 includes a thrombectomy or atherectomy catheter 12. The catheter 12 includes one or more sheaths. For example, the illustrative catheter 12 includes an outer sheath 14 that is coaxial with and surrounds and encloses an inner sheath 16 (referred to as a deployment sheath 16). The deployment sheath 16 includes a lumen 18 configured to house a tether 20.

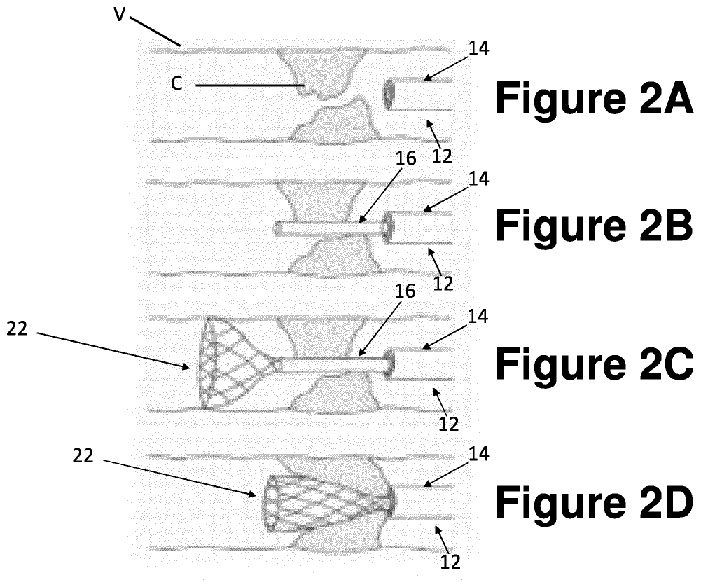

With continuing reference to FIG. 1 and with further reference to FIGS. 2A-2D, a capture device 22 is secured to an end of the tether 20, and is configured to be deployed to a clot in a blood vessel out of a distal end of the catheter 12 from the lumen 18 via the deployment sheath 16. FIG. 1 shows the capture device 22 in its deployed state located outside of the lumen 18. The capture device 22 of FIG. 1, when deployed and expanded, has a narrow end E_{N} located proximate to the end of the deployment sheath 16, and has an opposite wide end E_{W} located distal from the end of the deployment catheter 16. To initiate intravascular therapy using the intravascular therapy device 10, the distal end of the catheter 12 is inserted into a blood vessel (e.g., a vein in the case of a thrombectomy procedure, or an artery in the case of an atherectomy procedure) through an incision accessing a blood vessel made by the surgeon. A suitable length of the catheter 12 is then pushed through the blood vessel until the tip of the catheter 12 is positioned at the treatment site (see FIG. 2A). This may optionally be done with the aid of a suitable medical imaging modality such as ultrasound or computed tomography (CT) imaging to guide the surgeon in placement of the catheter tip.

During the insertion process, the expandable capture device 22 is compacted (i.e. compressed) inside the lumen 18 of the deployment sheath 16 so it does not interfere with movement of the catheter through the vasculature. Not shown in FIG. 1 is an opposite end of the catheter 12, which typically includes a handle with a deployment mechanism connected to the end of the tether 20 at the handle. The deployment mechanism is operated by the surgeon to push the tether 20 outward relative to the deployment sheath 16 to push the capture device 22 out of the lumen 18 and thereby deploy the capture device 22 in the blood vessel (see FIG. 2C). The capture device 22 is designed to expand when outside of the lumen 18, by being constructed as a self-expanding capture device 22 (e.g., being made of an elastically compressible material such as Nitinol) or by including an inflatable balloon (not shown) inside the capture device that is inflated at the appropriate time by flowing an inflation fluid through a lumen of the catheter.

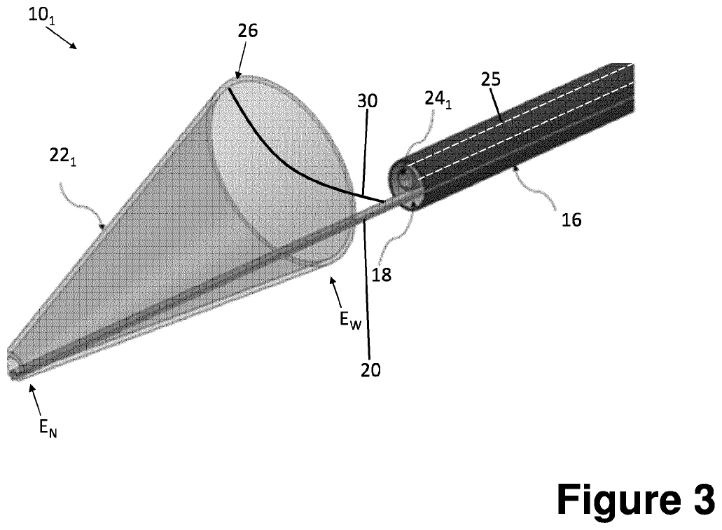

The tether 20 is also configured to draw the capture device 22 back into the lumen 18 of the deployment sheath 16 after deployment of the capture device 22. Again, this is typically done by operating a suitable control of the handle. The capture device 22 is configured to capture the material of the clot during the withdrawal process (see FIG. 2D). As further shown in the sequence of FIGS. 2B-2D, in some intravascular procedures the deployment and withdrawal process may be more complex, such as including movement of the deployment sheath 16 out of the tip of the outer sheath 14 (see FIG. 2B) prior to deploying the capture device 22 out of the deployment sheath 16 to strategically deploy the capture device 22 beyond the target clot. As shown in FIG. 1, the capture device 22 comprises a cage, but can also be any suitable expandable structure (e.g., a basket as shown in FIG. 3, a funnel, and so forth).

The foregoing process is sometimes referred to as a clot retrieval process. As the clot is withdrawn back into the lumen 18 of the deployment sheath 16 the goal is for the clot material captured by the capture device 22 to be drawn into the sheath by mechanical entanglement and transport by the capture device 22. Additionally, vacuum aspiration may be performed through the lumen 18 to suck the clot material through the lumen 18 back to a bag or other receptacle in or with the handle at the end of the catheter 14 opposite from the distal end shown in FIG. 1. However, as can be seen in FIG. 2D, this involves the clot material being compacted, mechanically and/or by aspiration, into the relatively narrow-diameter lumen 18. In this compaction process, it is possible for clot material to break away and flow downstream by action of the blood flow past the tip of the catheter 14. Such breakaway clot material portions then flows downstream through the vasculature as an embolus and can lodge in another area of the vasculature forming an embolism that may partially or completely block blood flow, creating a serious and undesirable medical complication.

In embodiments disclosed herein, laser ablation is used to ablate the captured clot material so as to avoid such generation of emboli. This approach differs from conventional laser ablation treatment of a clot because, rather than attempting to steer a laser aperture to the clot material disposed on the vessel wall in order to ablate it in situ, the capture device 22 is used to capture the clot material and draw it off the blood vessel wall and bring the captured clot material to the laser aperture. This approach transforms the problematic step of compacting the captured clot material into the relatively narrow lumen 18 of the deployment sheath 16, which in conventional approaches is prone to generating emboli, into an opportunity to move the clot material into the relatively short (e.g. tens of microns) working distance of the laser aperture to efficiently ablate the captured material. Moreover, because the clot material is brought to the laser aperture rather than attempting to steer the laser aperture to the clot material in its in situ position on the vessel wall, the disclosed approach eliminates the need for a complex multiple degrees-of-freedom aperture steering mechanism, instead leveraging the less mechanically complex deployment/retrieval mechanism of a clot retrieval device to bring the clot material into close proximity to the laser aperture for ablation.

To this end, FIG. 1 also shows that the device 10 includes a laser aperture 24 disposed proximate to the capture device 22, and configured to ablate material of a clot in a blood vessel of a patient that is captured by the capture device 22. The laser aperture 24 is arranged to ablate the clot material as it is mechanically transported to the lumen 18 of the intravascular clot retrieval device 14, 22. As shown in FIG. 1, the laser aperture 24 can comprise a ring of optical fiber ends disposed at a circumference of the distal end of the outer sheath 14, where the optical fibers themselves run through the length of the outer sheath 14 toward the handle and are connected with an excimer laser or other type of laser (not shown) located outside of the patient. In another example, the laser aperture 24 comprises an optical fiber or bundle of optical fibers disposed in the lumen 18 of the deployment sheath 16. In another example, the laser aperture 24 comprises an end or ends of one or more optical fibers integrated into a portion of the capture device 22. In another example, the laser aperture 24 comprises one or more laser fibers mounted in an interior of the capture device 22 (i.e., when the capture device 22 comprises a funnel). These are merely examples, and should not be construed as limiting.

FIG. 3 shows another example of a device 10₁. This embodiment retains the outer sheath 14, deployment sheath 16 with lumen 18, and tether 20 of the embodiment of FIG. 1. (Note, the outer sheath 14 is not shown in FIG. 3). However, a capture device 22₁ of FIG. 3 has an opposite orientation when deployed and expanded compared with the capture device 22 of FIG. 1, with its narrow end E_{N} located distal from the end of the deployment sheath 16 and its opposite wide end E_{W} located proximate to the end of the deployment sheath 16. Furthermore, the capture device 22₁ is arranged eccentrically respective to the deployment sheath 16 (and hence also arranged eccentrically respective to the outer sheath, not shown in FIG. 3 but arranged coaxially around the deployment sheath 16), which places an outer edge 26 of the wide end E_{W} of the capture device 22₁ further away from the common axis of the coaxial sheaths 14, 16. This provides the capture device 22₁ with a larger radial “reach” to capture clot material compared with the capture device 22 of FIG. 1, and hence may be particularly useful for removing clot material from larger-diameter blood vessel lumens. (Due to the eccentric arrangement of the capture device 22₁, multiple deployment/retrieval operations may need to be performed with the outer edge 26 at different angular positions around the common axis of the sheaths 16, 18).

The capture device 22₁ also advantageously is arranged to operate as a scoop, with clot material cleaved from the blood vessel wall by the outer edge 26 being funneled toward the narrow end E_{N} within the interior of the capture device 22₁ when it is retrieved back into the lumen 18. To retain the expanded capture device 22₁ in the eccentric arrangement, both the narrow end E_{N} and an edge 26 of the wide end E_{W} are suitably secured to the tether 20. To enable retrieval of the capture device 22₁ back into the narrow-diameter lumen 18 in spite of the wide end E_{W} facing the lumen 18, a wire or thread 30 loosely connects the outer edge 26 to the tether 20 as shown in FIG. 3. When the tether 20 is drawn back into the lumen 18, the wire or thread 30 is pulled back to the axis of the tether 20 thus operating to close the expanded open end E_{W} so the capture device 22₁ can be compacted back into the lumen 18 during the retrieval.

The clot retrieval device of FIG. 3 further includes a laser aperture 24₁ that is located differently from the laser aperture 24 of the embodiment FIG. 1. The illustrative laser aperture 24₁ comprises the end or ends of an optical fiber or fiber bundle 25 that passes through the lumen 18 alongside the tether 20. More particularly, the optical fiber or fiber bundle 25 is located on the same side of the tether 20 as the outer edge 26 of the wide end E_{W} of the capture device 22₁. In this arrangement, the laser aperture 24₁ is positioned to be close to the clot material that is captured in the interior of the capture device 22₁ as the capture device 22₁ is retrieved back into the lumen 18. In another example, the end or ends of an optical fiber or fiber bundle 25 can be disposed mounted in an interior of the capture device 22₁ (when the capture device 22₁ comprises a funnel).

It is to be appreciated that the intravascular clot retrieval devices 10, 10₁ of FIGS. 1 and 3 are illustrative examples, and that more generally substantially any type of intravascular clot retrieval device can be combined with a laser aperture arranged to ablate the clot material as it is mechanically transported to the lumen 18 of the intravascular clot retrieval device in accordance with the disclosure herein, so as to ablate captured clot material and thereby reduce or eliminate the likelihood of generating emboli.

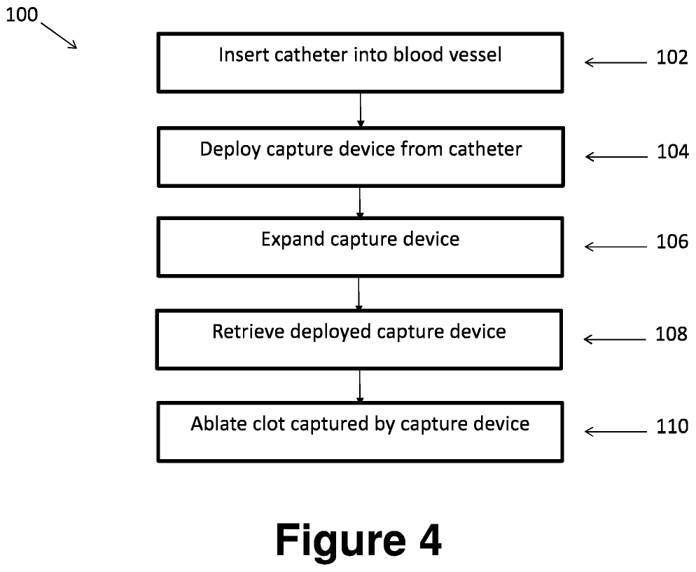

FIG. 4 shows an example of a flowchart showing a thrombectomy or atherectomy method 100 using the device 10 of FIG. 1 or the device 10₁ of FIG. 3. Reference is also made back to FIGS. 2A-2D which show steps of the operations of the method 100 for the illustrative case of using device 10 of FIG. 1. At an operation 102, the catheter 12 (carrying the expandable capture device 22) is inserted into a blood vessel V to deliver the capture device 22 to a clot C disposed on an inner wall of the blood vessel V. As shown in FIG. 2A, the catheter 12 is disposed on a “right” side of the clot C (referring to the illustrative orientation depicted in FIGS. 2A-2D).

At an operation 104, the expandable capture device 22 is deployed from the lumen 18 of the deployment sheath 16. As shown in FIG. 2B, the deployment sheath 16 carrying the compacted capture device 22 (not seen in FIG. 2B) is advanced from the outer sheath 14 until a distal end of the deployment sheath 16 is disposed on a “left” side of the clot C.

At an operation 106, the capture device 22 is deployed. As shown in FIG. 2C the expandable capture device 22 is moved out of the end of the deployment sheath 16 and expands (i.e., to expand the cage of the capture device 22). In some embodiments, the capture device 22 is a self-expanding device, for example made of a material such as Nitinol that is compacted into the lumen 18 and hence self-expands when pushed outside of the lumen 18. In other embodiments, an inflatable balloon (not shown) may be disposed inside the capture device and inflated to expand the capture device. As can be seen in FIG. 2C, the expanded capture device 22 is on an opposite side of the clot C from the end of the outer sheath 14, and the deployment sheath 16 extends through the clot C.

At an operation 108, the deployed expandable capture device 22 is retrieved by the tether 20 back into the lumen 18 of the deployment sheath 16. As shown in FIG. 4D, this operation in the illustrative example includes both withdrawing the deployment sheath 16 back through the clot C and back into the outer sheath 14, and retrieving the capture device 22 back into the lumen 18 of the deployment sheath 16. As the capture device 22 is retrieved, it grabs at least a portion of the clot C, and mechanically transports the captured clot material toward the lumen 18.

At an operation 110 (which can be performed during the deploying operation 106 and/or during the retrieving operation 108), material of the clot C that is captured by the capture device 22 is ablated with laser light emitted by the laser aperture 24. The retrieval of the material of the clot C captured by the capture device 22 is moved towards the laser aperture 24 during the retrieval of the capture device 22, thus moving the clot material within the relatively short working distance of the laser aperture 24 to effectuate the ablation of the captured material with laser light emitted by the laser aperture 24. The ablation of the captured material of the clot C occurs within a working distance of typically 200 microns or less from the laser aperture 24, and more typically within about 50 microns or less. Ordinarily, such laser apertures are brought to the clot, which requires bringing the laser aperture nearly into contact with the clot, requiring complex manipulation of the tip carrying the aperture by a medical professional. Advantageously, the material of the clot C in the approach of FIGS. 2 and 4 is brought to the laser aperture 24 of the device 10.

In a variant embodiment (not shown), the laser aperture may be integrated with the expandable capture device itself. For example, referring back to the embodiment of FIG. 1, in place of the illustrative laser aperture 24 a set of optical fibers can be secured with the tether 20 as a bundle running through the lumen 18, and the optical fibers can then split off at the junction of the narrow end E_{N} of the capture device 22 and run along the surface of the capture device 22 toward the wide end E_{W}, with the fiber ends being located at selected points on the surface of the capture device 22. In this case, the laser ablation of the captured clot material may occur at any time during the deployment and subsequent retrieval of the capture device 22 that the capture device 22 is engaged with said clot material. In an analogous variant embodiment starting from the embodiment of FIG. 3, the laser aperture 24₁ is replaced by a set of optical fibers secured with the tether 20 as a bundle running through the lumen 18, and the optical fibers run to the end of the tether 20 where it connects with the narrow end E_{N} of the capture device 22₁ so that the laser aperture is located at the narrow end E_{N} of the capture device 22₁. In this case, the laser ablation of the captured clot material may occur at any time during the deployment and subsequent retrieval of the capture device 22 that clot material is funneled to the narrow end E_{N}.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A thrombectomy or atherectomy device, comprising:
a catheter;
a capture device disposed at a distal end of the catheter, said capture device comprising a distal end having a first width and a proximal end having a second width wherein said first width is different from said second width; and
a laser aperture disposed proximate to the capture device and configured to ablate material of a clot in a blood vessel of a patient that is captured by the capture device.

2. The device of claim 1, wherein the capture device comprises a self-expanding Nitinol device.

3. The device of claim 1, wherein the capture device comprises an expandable structure configured to capture the material of the clot.

4. The device of claim 3, wherein the expandable structure comprises a cage or a basket.

5. The device of claim 3, wherein the catheter comprises a deployment sheath configured to deploy the capture device from a lumen of the deployment sheath.

6. The device of claim 5, wherein the catheter further comprises an outer sheath configured to enclose the deployment sheath.

7. The device of claim 5, wherein the laser aperture comprises a ring of optical fiber ends disposed at a circumference of the distal end of the outer sheath.

8. The device of claim 5, wherein the catheter further comprises:
a tether passing through the lumen of the deployment sheath and secured to the capture device to draw the capture device back into the lumen of the deployment sheath after deployment of the capture device; and
an optical fiber or bundle of optical fibers disposed in the lumen of the deployment sheath wherein an end of the optical fiber or bundle of optical fibers defines the laser aperture.

9. The device of claim 8, wherein the capture device is configured to expand eccentrically respective to a center of the deployment sheath.

10. The device of claim 1, wherein the laser aperture comprises an end or ends of one or more optical fibers integrated into a portion of the capture device.

11. The device of claim 1, wherein the capture device comprises a funnel.

12. The device of claim 11, wherein the laser aperture comprises one or more laser fibers mounted in an interior of the funnel.

13. A thrombectomy or atherectomy method comprising:
inserting a catheter carrying an expandable capture device into a blood vessel to deliver the capture device to a clot disposed on an inner wall of the blood vessel;
deploying the expandable capture device from a lumen of the catheter;
retrieving the deployed expandable capture device back into the lumen of the catheter; and
during the deploying and/or during the retrieving, ablating material of the clot that is captured by the capture device with laser light emitted by a laser aperture disposed at a distal end of the catheter or on the capture device.

14. The method of claim 13, wherein the laser aperture is disposed at the distal end of the catheter, and the retrieving moves the material of the clot that is captured by the capture device toward the laser aperture to effectuate the ablation of the captured material with the laser light.

15. The method of claim 14, wherein the ablation of the captured material occurs within a working distance of 200 microns or less from the laser aperture.

16. The method of claim 13, wherein the laser aperture is disposed on the capture device.

17. A thrombectomy or atherectomy device, comprising:
an intravascular clot retrieval device configured to mechanically transport clot material to a lumen of the intravascular clot retrieval device; and
a laser aperture arranged to ablate the clot material as it is mechanically transported to the lumen of the intravascular clot retrieval device.

18. The thrombectomy or atherectomy device of claim 17, wherein the intravascular clot retrieval device comprises:
an expandable capture device; and
a catheter including a deployment sheath having a lumen receiving the expandable capture device and a retrieval tether passing through the lumen and secured to the expandable capture device, the catheter being configured to deploy the expandable capture device out of the lumen and to subsequently retrieve the deployed expandable capture device back into the lumen;
wherein the laser aperture is arranged at a distal end of the catheter to apply ablating laser light to the expandable capture device as the expandable capture device is retrieved back into the lumen.

19. The thrombectomy or atherectomy device of claim 18, wherein the catheter further includes an outer sheath surrounding the deployment sheath and the laser aperture comprises a laser aperture ring disposed on the outer sheath.

20. The thrombectomy or atherectomy device of claim 18, wherein the laser aperture comprises an end of an optical fiber or optical fiber bundle disposed in the lumen.
